(19) United States
(12) Patent Application Publication
(10) Pub. No.: US 2019/0051405 A1
(43) Pub. Date: Feb. 14, 2019

(54) DATA GENERATION APPARATUS, DATA GENERATION METHOD AND STORAGE MEDIUM

(71) Applicant: FUJITSU LIMITED, Kawasaki-shi (JP)

(72) Inventors: Kentaro DOI, Kawasaki (JP); Kouhei SHIGETA, Yokohama (JP)

(73) Assignee: FUJITSU LIMITED, Kawasaki-shi (JP)

(21) Appl. No.: 16/054,558

(22) Filed: Aug. 3, 2018

(30) Foreign Application Priority Data
Aug. 9, 2017 (JP) 2017-154679

(51) Int. Cl. G16H 40/20; G16H 50/30; G16H 50/70; G16H 10/60

(52) U.S. Cl. CPC G16H 40/20; G16H 10/60; G16H 50/70; G16H 50/30

(57) ABSTRACT

A non-transitory computer-readable storage medium storing a program that causes a computer to execute a process, the process includes accepting a designation of an inspection item; acquiring, from inspection result information in which inspection results and inspection date and time of a patient are stored, inspection results and inspection date and time corresponding to the designated inspection item; referring to period information that defines a period within which, when a treatment is performed for the patient, an inspection result of an inspection item corresponding to a treatment is not used for generation of prediction data, intervention information in which a processing substance carried out for the patient and treatment date and time are stored, and inspection date and time for each of the acquired inspection results, and specifying inspection results for generation of the prediction data; and generating the prediction data based on the specified inspection results.

### CROSS-REFERENCE TO RELATED APPLICATION

This application is based upon and claims the benefit of priority of the prior Japanese Patent Application No. 2017-154679, filed on Aug. 9, 2017, the entire contents of which are incorporated herein by reference.

### FIELD

The embodiment discussed herein relates to a data generation apparatus, a data generation method and a storage medium.

### BACKGROUND

In a hospital, a patient sometimes undergoes a same inspection by a plural number of times. A result of the inspection is stored in an associated relation with identification information of the patient, an inspection type and inspection date and time into an electronic medical record database (DB). The interval between inspections of each patient fluctuates depending upon the patient's convenience, a doctor's policy or the like.

On the other hand, in the case where a patient undergoes medical care (treatment) such as treatment of disease or surgical operation, the substance of the treatment in a hospital, date and time of the treatment and so forth are stored in an associated relation with the identification information of the patient into the electronic medical record DB. As a related art, for example, Japanese Laid-open Patent Publication No. 2016-126718 and so forth are disclosed.

In the case where a certain patient undergoes an inspection, it is sometimes desired to predict the patient condition in the future based on the result of the inspection. In this case, preferably the patient condition in the future may be predicted with a high degree of accuracy utilizing the data stored in the electronic medical record DB described above. In view of the above, it is desirable to be capable of generating prediction data for predicting the patient condition.

### SUMMARY

According to an aspect of the embodiment, a non-transitory computer-readable storage medium storing a program that causes a computer to execute a process, the process includes accepting a designation of an inspection item; acquiring, from inspection result information in which inspection results and inspection date and time of a patient are stored, inspection results and inspection date and time corresponding to the designated inspection item; referring to period information that defines a period within which, when a treatment is performed for the patient, an inspection result of an inspection item corresponding to the treatment is not used for generation of prediction data, intervention information in which a processing substance carried out for the patient and treatment date and time are stored, and inspection date and time for each of the acquired inspection results, and specifying inspection results to be used for generation of the prediction data from among the acquired inspection results; and generating the prediction data based on the specified inspection results.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a view schematically depicting a configuration of a medical system according to an embodiment;

FIG. 2A is a view depicting a hardware configuration of an electronic medical record server and a prediction server;

FIG. 2B is a view depicting a hardware configuration of a doctor terminal and a manager terminal;

FIG. 3 is a functional block diagram of a prediction server;

FIG. 4A is a view depicting an example of a data structure of an inspection result table;

FIG. 4B is a view depicting an example of a data structure of an intervention table;

FIG. 4C is a view depicting an example of a data structure of a period master;

FIGS. 5A and 5B are flow charts depicting an example of a generation process of prediction data by a prediction server;

FIGS. 6A, 6B and 6C are views illustrating the process of FIGS. 5A and 5B;

FIG. 7 is a view depicting an example of a data structure of a prediction data DB;

FIG. 8 is a flow chart depicting an example of a prediction process by a prediction unit;

FIG. 9 is a view for explaining about a learned model; and

FIG. 10 is a view depicting a modification to the period master.

### DESCRIPTION OF EMBODIMENT

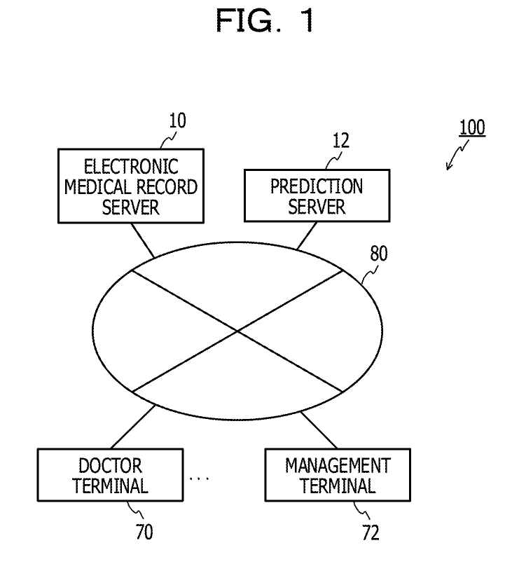

In the following, an embodiment of a medical system is described in detail with reference to FIGS. 1 to 9.

FIG. 1 schematically depicts a configuration of a medical system according to the embodiment. As depicted in FIG. 1, the medical system 100 includes an electronic medical record server 10, a prediction server 12, a doctor terminal 70 and a manager terminal 72. The electronic medical record server 10, prediction server 12 as a data generation apparatus, doctor terminal 70 and manager terminal 72 are coupled to a network 80 such as the Internet or a local area network (LAN).

The electronic medical record server 10 retains an electronic medical record DB and manages data relating to patients themselves, data relating to inspections carried out for the patients, data relating to treatments (medical treatments, surgical operations and so forth) carried out for the patients, data relating to patient's symptoms and so forth. The data relating to each patient itself includes identification information, name, sex, age, birth date, height, weight and so forth of the patient. The data relating to each inspection includes an inspection type, inspection date and time, an inspection result and so forth. The data relating to each treatment includes the substance of the treatment, date and time of the treatment and so forth. The data relating to each symptom includes the substance of the symptom, date and time when the symptom develops, data and time when the symptom ends and so forth. Such pieces of data stored in the electronic medical record server 10 are data suitably inputted by a doctor or the like through the doctor terminal 70 or the like.

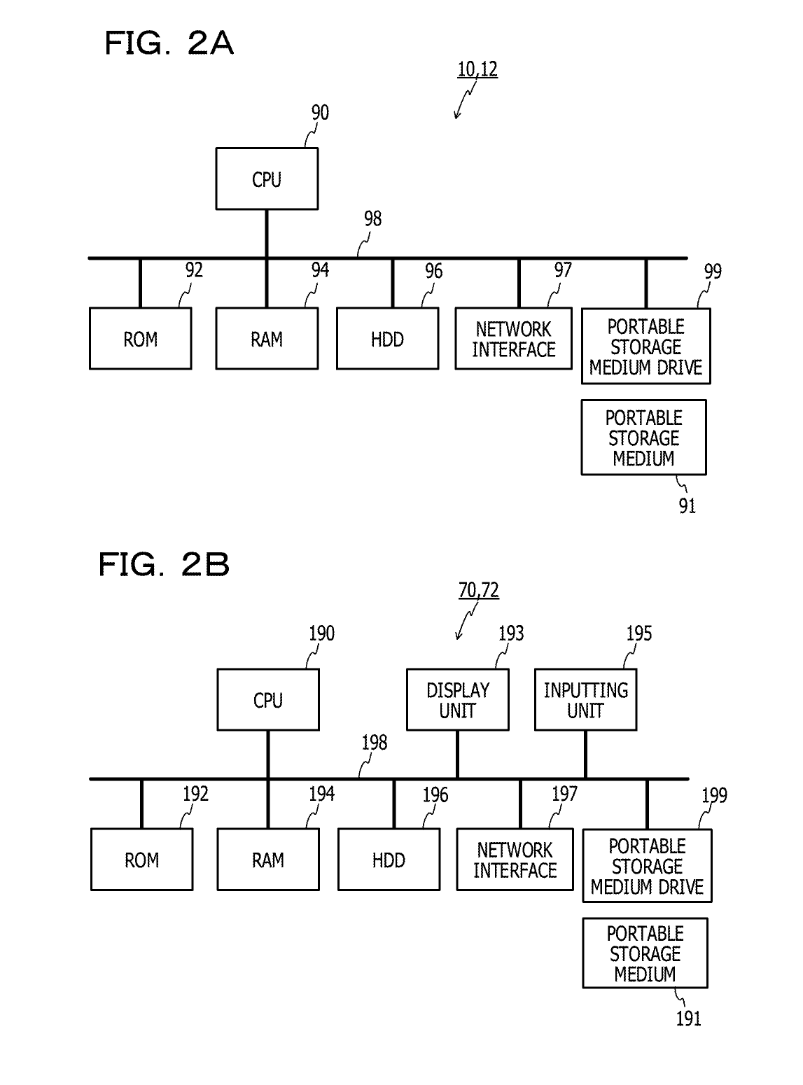

FIG. 2A depicts an example of a hardware configuration of the electronic medical record server 10. As depicted in FIG. 2A, the electronic medical record server 10 includes a central processing unit (CPU) 90, a read only memory (ROM) 92, a random access memory (RAM) 94, a storage unit (here, a hard disk driver (HDD)) 96, a network interface 97, a portable storage medium drive 99 that may read a program or data stored in a portable storage medium 91, and so forth. In the HDD 96, an electronic medical record DB is stored. The components mentioned of the electronic medical record server 10 are coupled to a bus 98.

The prediction server 12 uses the data accumulated in the electronic medical record server 10 to generate prediction data to be used to predict a future state of a patient who has undergone an inspection. The prediction server 12 generates a learned model hereinafter described from the prediction data. Then, the prediction server 12 predicts a future state of the patient who has undergone an inspection based on the generated learned model and an inspection result of the patient who has undergone the inspection.

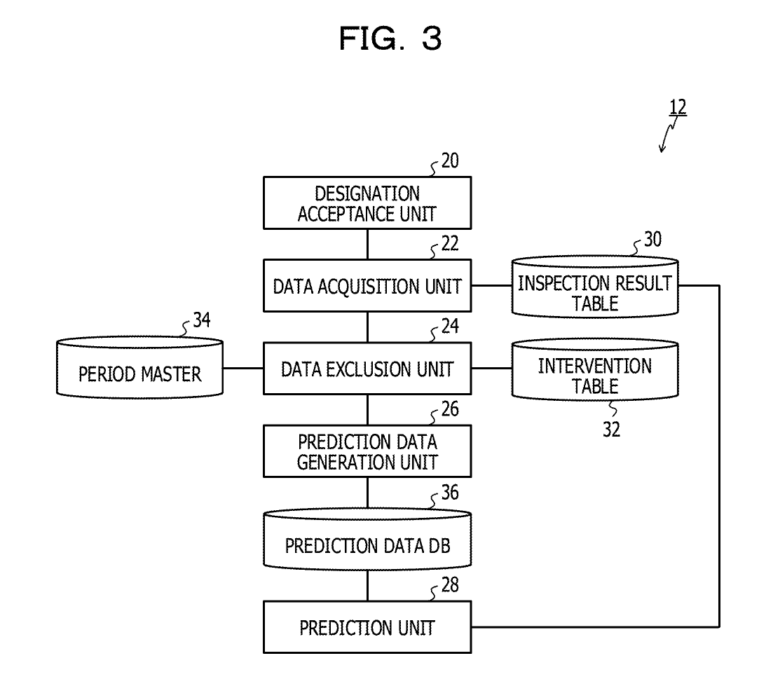

The prediction server 12 includes such a hardware configuration as depicted in FIG. 2A similarly to the electronic medical record server 10. In the prediction server 12, the CPU 90 executes a program (including a data generation program) stored in the ROM 92 or the HDD 96 or a program (including a data generation program) read from the portable storage medium 91 by the portable storage medium drive 99 to implement functions of the components depicted in FIG. 3

FIG. 3 depicts a functional block diagram of the prediction server 12. As depicted in FIG. 3, as the CPU 90 executes a program, the prediction server 12 functions as a designation acceptance unit 20, a data acquisition unit 22 as an acquisition unit, a data exclusion unit 24 as a specification unit, a prediction data generation unit 26 as a generation unit and a prediction unit 28.

The designation acceptance unit 20 accepts an inspection type designated by a manager or a doctor and information of a symptom of a patient through the manager terminal 72 or the doctor terminal 70 and passes the accepted information to the data acquisition unit 22. In the case where the manager or the doctor wants to generate prediction data, the manager or the doctor designates which inspection result for a patient with whom a certain symptom has appeared is to be used to generate prediction data through the manager terminal 72 or the doctor terminal 70. For example, in the case where the manager or the doctor wants to generate prediction data using an inspection result of the “cell mass” of a patient who has developed “tuberculosis,” the manager or the doctor designates the symptom “tuberculosis” and the inspection result “cell mass.”

The data acquisition unit 22 acquires data to be used to generate prediction data from an inspection result table 30 as a first storage unit based on the information received from the designation acceptance unit 20.

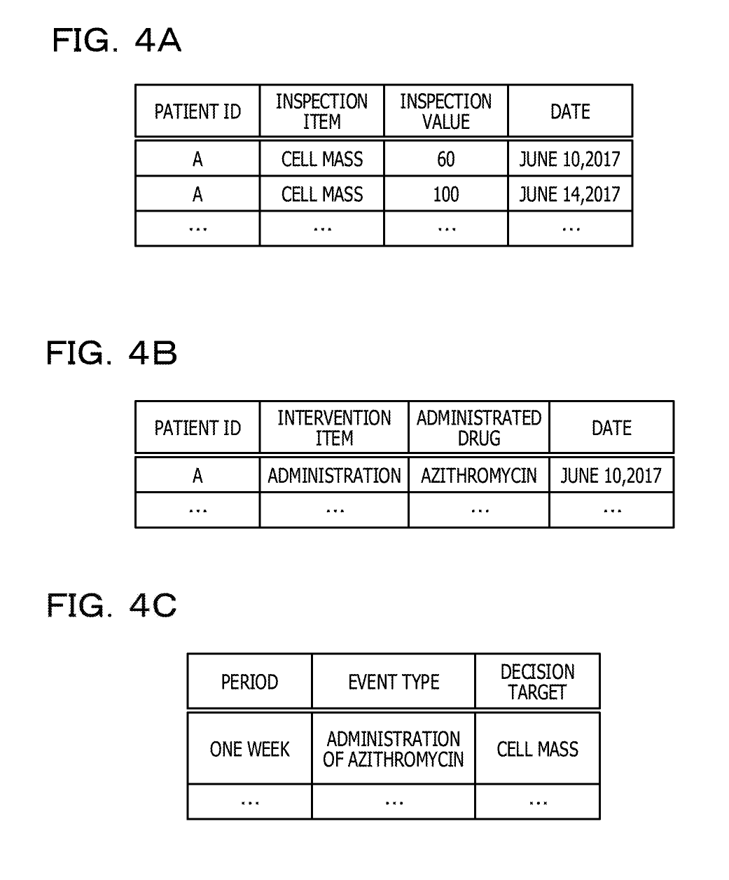

Here, the inspection result table 30 includes such a data structure as depicted in FIG. 4A. For example, the inspection result table 30 includes fields for “patient ID,” “inspection item,” “inspection value” and “date” as depicted in FIG. 4A. In the field for “patient ID,” identification information of a patient is placed. In the field for “inspection item,” information relating to an inspection type is placed. In the field for “inspection value,” a value of an inspection result is placed. In the field for “date,” information of inspection date and time is placed. In the inspection result table 30, only data corresponding to these fields from among all data managed in the electronic medical record server 10 is replicated.

Accordingly, the data acquisition unit 22 specifies identification information (patient ID) of a patient in whom a designated symptom has developed from the data managed in the electronic medical record server 10. Then, the data acquisition unit 22 acquires an inspection result and inspection date and time of the designated inspection type of the specified patient from the inspection result table 30.

The data exclusion unit 24 refers to a period master 34 as a definition unit. Then, the data exclusion unit 24 excludes, based on data stored in an intervention table 32 as a second storage unit, data that are not to be used for generation of prediction data from among the data acquired from the inspection result table 30 and specifies only the data to be used for generation of prediction data.

Here, the intervention table 32 has stored therein information relating to treatments (surgical operations, administration and so forth) executed for patients and includes such a data structure as depicted in FIG. 4B. For example, as depicted in FIG. 4B, the intervention table 32 includes fields for “patient ID,” “intervention item,” “administrated drug” and “date.” In the field for “patient ID,” identification information of a patient who has undergone a treatment is placed, and in the field for “intervention item,” an item name relating to the treatment (“surgical operation,” “administration” or the like) is placed. In the field for “administrated drug,” the name of an administrated drug is placed. In the field for “date,” information of the treatment date and time is placed. In the intervention table 32, only data corresponding to these fields from among all data managed in the electronic medical record server 10 is replicated.

The period master 34 is a master that defines a period within which, when a treatment is performed for a patient, an inspection result of an inspection item corresponding to the treatment is not used for generation of prediction data and includes such a data structure as depicted in FIG. 4C. For example, as depicted in FIG. 4C, the period master 34 includes fields for “period,” “event type” and “decision target.” In the field for “period,” information of a period within which the inspection result is not used for generation of prediction data is placed, and in the field for “event type,” the substance of the treatment (information including an intervention item and an administrated drug of FIG. 4B) is placed. In the field for “decision target,” which inspection item is to be invalidated is placed. In the example of FIG. 4B, it is defined that an inspection result of an inspection of the cell mass that has been undergone by a patient, to whom azithromycin was administrated, within one week after the administration is not used for generation of prediction data. The reason why such definition is made is that, since the cell mass decreases by an influence of azithromycin for one week after azithromycin was administrated, it is not appropriate to use the inspection result of the cell mass obtained within the period for generation of prediction data.

The prediction data generation unit 26 generates prediction data using the data specified by the data exclusion unit 24 to be used for generation of prediction data. The prediction data generation unit 26 stores the generated prediction data into a prediction data DB 36. Details of the prediction data and details of the data structure of the prediction data DB 36 are hereinafter described.

The prediction unit 28 acquires, based on the patient ID of the patient inputted from the doctor terminal 70 (patient of a prediction target), inspection results of inspections undergone till then by the patient of the prediction target from the inspection result table 30. The prediction unit 28 acquires prediction data corresponding to the acquired inspection results from the prediction data DB 36 and generates a learned model using the acquired prediction data. Then, the prediction unit 28 predicts a future state of the patient of the prediction target based on the generated learned model and the acquired inspection results.

Referring back to FIG. 1, the doctor terminal 70 is a terminal used by a doctor and includes such a hardware configuration as depicted in FIG. 2B. For example, the doctor terminal 70 includes a CPU 190, a ROM 192, a RAM 194, a storage unit (here, an HDD) 196, a network interface 197, a display unit 193, an inputting unit 195, a portable storage medium drive 199 that may read a program or data stored in a portable storage medium 191, and so forth. The components of the doctor terminal 70 are coupled to a bus 198. The display unit 193 includes a liquid crystal display or the like, and the inputting unit 195 includes a keyboard, a mouse, a touch panel and so forth. A doctor inputs various data to be managed by the electronic medical record server 10 and confirms various data managed by the electronic medical record server 10 through the doctor terminal 70. The doctor issues a generation instruction of prediction data (designation of a symptom or an inspection type) or issues an instruction for predicting a future state of the patient to the prediction server 12 through the doctor terminal 70.

The manager terminal 72 is a terminal used by a manager of the medical system 100. The manager terminal 72 includes such a hardware configuration as depicted in FIG. 2B similarly to the doctor terminal 70. The manager issues a generation instruction of prediction data (designation of a symptom or an inspection type) to the prediction server 12 through the manager terminal 72. The manager inputs various kinds of information for setting a period master 34 through the manager terminal 72.

(Generation Process of Prediction Data by Prediction Server)

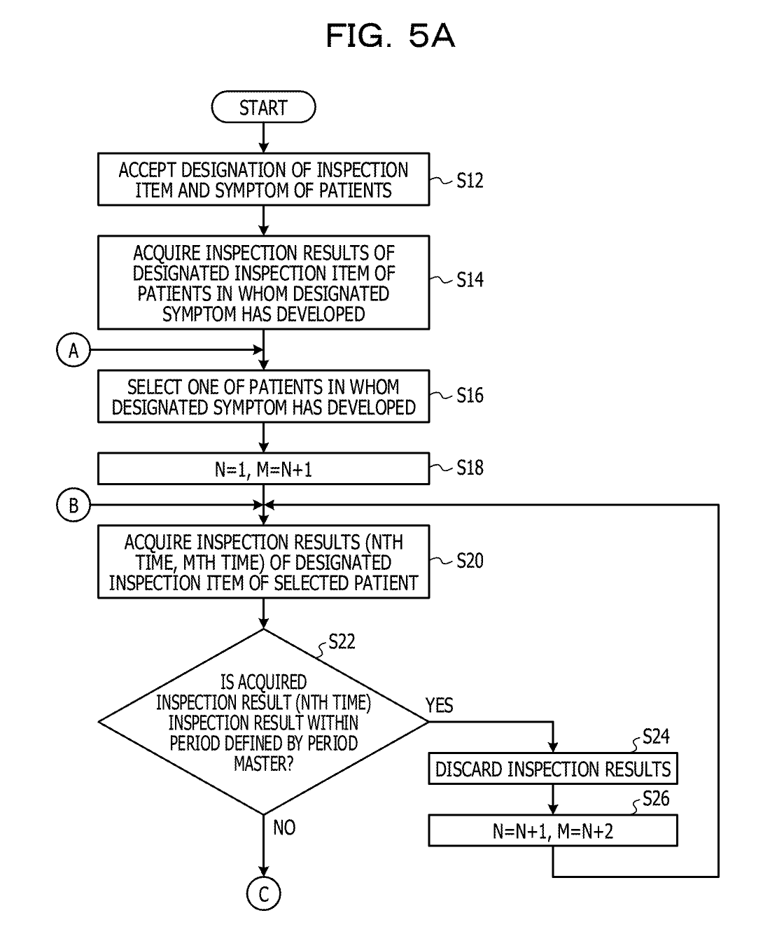

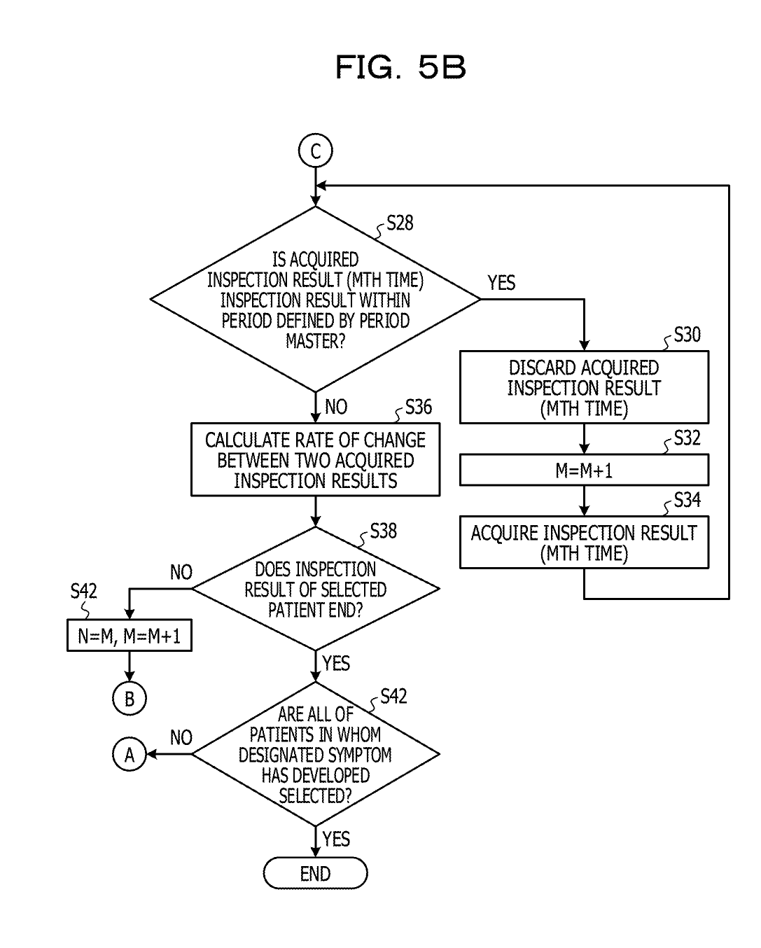

Now, a generation process of prediction data by a prediction server is described in detail with reference to flow charts of FIGS. 5A and 5B, and other figures. The prediction server mentioned in FIGS. 5A and 5B may be the prediction server 12 depicted in FIG. 1.

The process of FIGS. 5A and 5B is started at a stage at which, for example, the manager inputs a starting request for a generation process of prediction data through the manager terminal 72. At the point of time of starting of the process of FIGS. 5A and 5B, the period master 34 of FIG. 4C is generated already by the manager. Further, the data managed by the electronic medical record server 10 is replicated in the inspection result table 30 and the intervention table 32.

In the process of FIGS. 5A and 5B, first at S12, the designation acceptance unit 20 accepts a designation of an inspection item and a symptom of patients inputted from the manager terminal 72. The designation acceptance unit 20 passes the accepted information of the inspection item and the symptom to the data acquisition unit 22.

At S14, the data acquisition unit 22 acquires inspection results of the designated inspection item of the patients in whom the designated symptom has developed from the inspection result table 30. For example, if the designated symptom is “tuberculosis” and the designated inspection item is “cell mass,” the data acquisition unit 22 specifies patients in whom the symptom “tuberculosis” has developed in the electronic medical record server 10 and acquires all inspection results of “cell mass” of the specified patients from the inspection result table 30.

Then at S16, the data exclusion unit 24 selects one of the patients in whom the designated symptom has developed. Here, it is assumed that, for example, the patient of the patient ID=“A” is selected. In the following description, the patient of the patient ID=“A” is referred to as “patient A.”

Then at S18, the data exclusion unit 24 sets parameters N and M for the number of performed inspections to N=1 and M=N+1=2, respectively. The number of performed inspections signifies a turn number of an inspection performed for one patient from the oldest inspection. The oldest inspection is represented as “first time” and the second oldest inspection is represented as “second time,” for example.

Then at S20, the data exclusion unit 24 acquires inspection results (Nth time and Mth time) of the designated inspection item of the selected patient. Here, the data exclusion unit 24 acquires the inspection result of the cell mass of the patient A for the first time and the inspection result of the cell mass of the patient A for the second time.

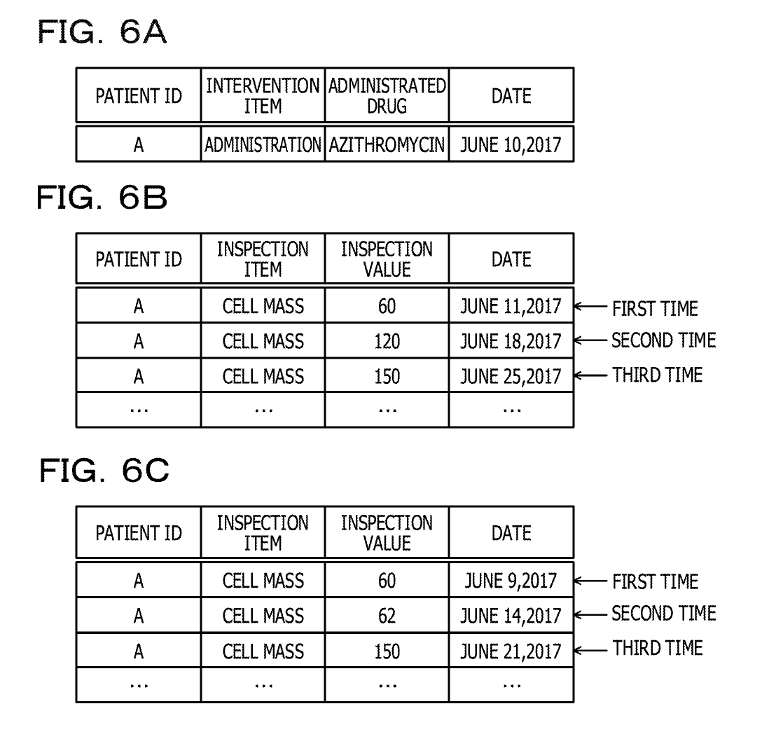

Then at S22, the data exclusion unit 24 decides whether or not the acquired inspection result (Nth time) is an inspection result within a period defined by the period master 34. FIGS. 6A, 6B and 6C are views illustrating the process of FIGS. 5A and 5B. For example, it is defined in the period master 34 as depicted in FIG. 4C that, within a period of time of one week after administration of azithromycin, an inspection result of the cell mass is invalidated, and it is assumed that azithromycin was administrated on Jun. 10, 2017 as depicted in FIG. 6A. In this case, if the Nth time (first time) inspection of the cell mass was performed on Jun. 11, 2017 as depicted in FIG. 6B, since the date is included in the period of invalidity described above, the decision at S22 is in the affirmative. In the case where the decision at S22 is in the affirmative, the processing advances to S24, at which the data exclusion unit 24 discards the acquired inspection results (Nth time and Mth time) once. In the case where the patient has undergone a plurality of treatments, if one of the inspection results is included in one of periods defined by the period master 34 corresponding to the treatments, the decision at S22 is in the affirmative.

Then at S26, the data exclusion unit 24 increments N and M by one (N=N+1, M=M+1=N+2). For example, if the decision at S22 is in the affirmative when N=1 and M=2 as in the example of FIG. 6B, at S26, the data exclusion unit 24 increments N and M to N=2 and M=3, respectively. Then, after the process at S26 is performed, the processing returns to S20. After the processing returns to S20, the data exclusion unit 24 acquires new Nth and Mth inspection results (here, the second and third time inspection results) and executes the decision at S22 similarly as described above.

On the other hand, in the case where the inspection of the cell mass for the first time was carried out on Jun. 9, 2017 as depicted in FIG. 6C, since the date does not included in the period of invalidity, the decision at S22 is in the negative, and the processing advances to S28.

The following description is given of a case in which the processing advances to S28 in a state in which the first time inspection result is not discarded and inspection results for the first and second times are acquired as depicted in FIG. 6C.

After the processing advances to S28, the data exclusion unit 24 decides whether or not the acquired inspection result (Mth time) is an inspection result within the period defined by the period master 34. In the case of FIG. 6C, the Mth time inspection result signifies the inspection result for the second time. In this case, since Jun. 14, 2017 on which the second time inspection was performed is included in the period of invalidity of the period master 34, the decision at S28 is in the affirmative, and the processing advances to S30. After the processing advances to S30, the data exclusion unit 24 discards the acquired inspection result (Mth time). In the example of FIG. 6C, the data exclusion unit 24 discards the inspection result for the second time. Then at S32, the data exclusion unit 24 increments M by one (M=M+1). In the example of FIG. 6C, M becomes M=3. Then at next S34, the data exclusion unit 24 acquires the inspection result (Mth time). In this case, a next inspection result (in FIG. 6C, an inspection result for the third time) to the inspection result discarded at S30 is acquired. Thereafter, the processing returns to S28. After the processing returns to S28, the data exclusion unit 24 decides whether or not the inspection result for the Mth time (=third time) is within the period of invalidity of the period master 34. However, in the example of FIG. 6C, the date is after the period of invalidity, and therefore, the decision at S28 becomes negative and the processing advances to S36.

After the processing advances to S36, the prediction data generation unit 26 calculates a rate of change between the two inspection results acquired at the current point of time (in FIG. 6C, between the inspection results for N=first time and M=third time). For example, the prediction data generation unit 26 determines the rate of change in accordance with the following expression (1):

rate of change=(Mth time inspection result−Nth time inspection result)/(number of days elapsed) (1)

The number of days elapsed signifies the number of days between the Nth time inspection and the Mth time inspection.

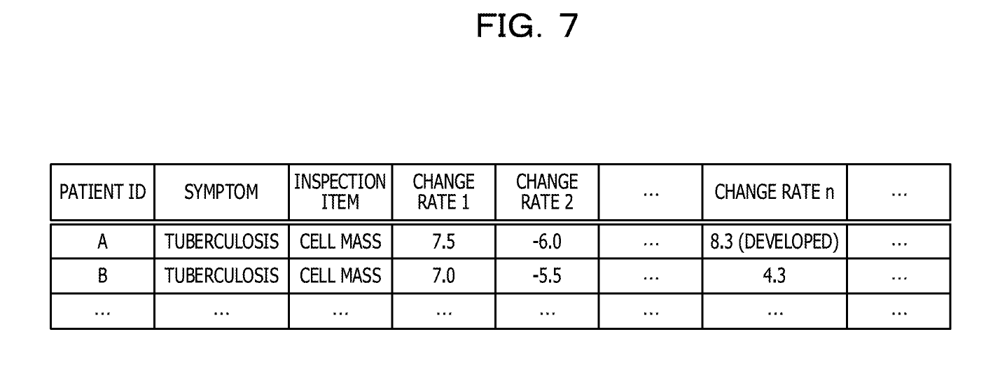

The prediction data generation unit 26 stores the determined rate of change into the prediction data DB 36. Here, the prediction data DB 36 includes such a data structure as depicted in FIG. 7. For example, the prediction data DB 36 is a database for storing data indicative of a symptom and an inspection item of each patient and a transition of the rate of change. The prediction data DB 36 includes fields for “patient ID,” “symptom,” “inspection item,” “change rate 1,” “change rate 2,” and the like. The prediction data generation unit 26 refers to the data managed in the electronic medical record server 10 to specify a date on which the symptom developed and stores “(developed)” together with the numerical value of the rate of change into the field for the rate of change immediately before the symptom developed.

Then at S38, the data exclusion unit 24 decides whether or not the inspection result of the selected patient comes to an end, namely, whether or not the data exclusion unit 24 has acquired all inspection results of the selected patient. If the decision at S38 is in the negative, then the processing advances to S40. After the processing advances to S40, the data exclusion unit 24 sets N and M to N=M and M=M+1, respectively. In the example of FIG. 6C, N and M become N=3 and M=4, respectively. Thereafter, the processing advances to S20, at which the data exclusion unit 24 acquires inspection results for the third and fourth times, whereafter the data exclusion unit 24 executes the processes at and after S22. In the case where the processing advances to S22 after it passes S40, the decision at S22 is in the negative without fail (because the decision at S28 executed immediately before then is in the negative). Accordingly, after the processing passes S40, the decision at S22 may be omitted.

On the other hand, if the decision at S38 is in the affirmative, the processing advances to S42, at which the data exclusion unit 24 decides whether or not all of the patients in whom the designated symptom has developed are selected. In the case where the decision at S42 is in the negative, the processing returns to S16, at which the data exclusion unit 24 selects a next patient, whereafter it repetitively executes the processes at and after S18. On the other hand, in the case where the decision at S42 is in the affirmative, the processes of the flow chart of FIGS. 5A and 5B are ended.

As described above, by executing the process of FIGS. 5A and 5B, the prediction server 12 stores transition data of the rate of change of the inspection result of the designated inspection item of each patient in whom the designated symptom has developed into the prediction data DB 36. In this case, in the case where a treatment that has an influence on the inspection result is performed, the rate of change of the inspection result is determined excluding the inspection results within a period within which the influence of the treatment appears. Accordingly, appropriate data from which the influence of the treatment is excluded and that indicates a transition of the rate of change of the inspection result may be stored into the prediction data DB 36.

(Process of Prediction Unit)

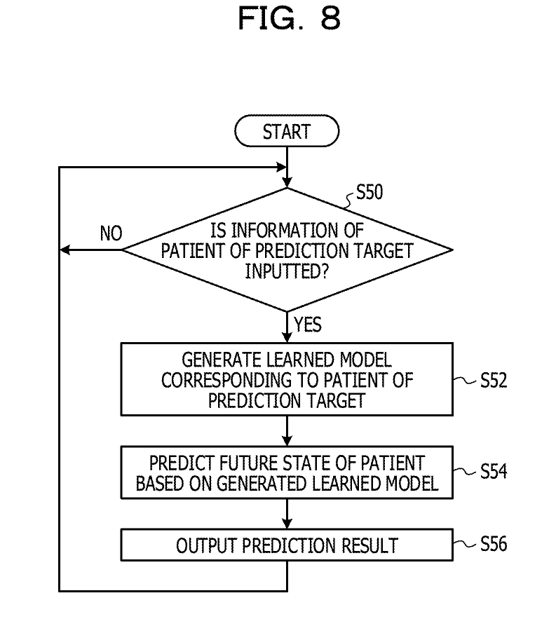

FIG. 8 depicts a process of a prediction unit in the form of a flow chart. The prediction unit mentioned in FIG. 8 may be the prediction unit 28 depicted in FIG. 3.

As depicted in FIG. 8, the prediction unit 28 stands by until information of a patient of a prediction target is inputted at S50. If a doctor designates a patient of a prediction target on the doctor terminal 70, the information of the designated patient is inputted from the doctor terminal 70 to the prediction unit 28. After the information of the patient is inputted in this manner, the prediction unit 28 advances the processing to S52.

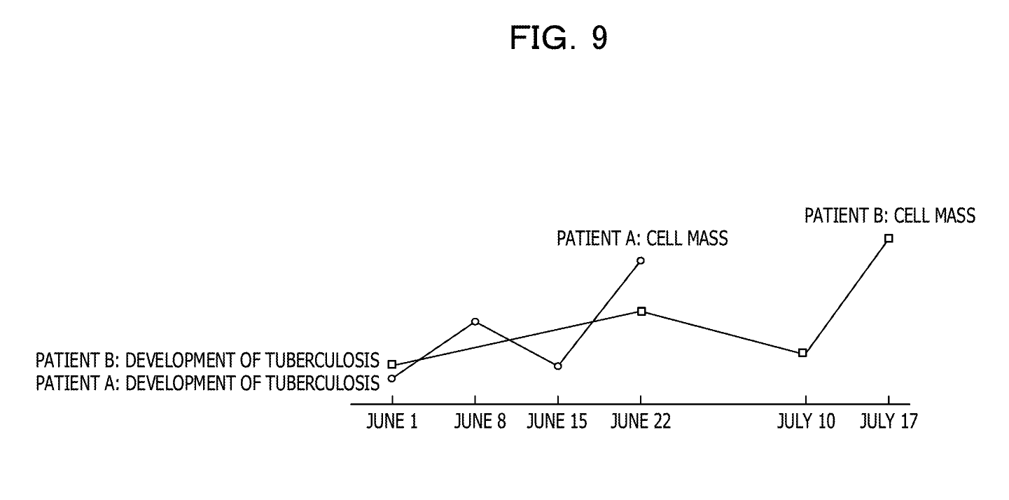

After the processing advances to S52, the prediction unit 28 refers to the prediction data DB 36 to generate a learned model corresponding to the patient of the prediction target. In this case, the prediction unit 28 reads out prediction data of patients who have undergone an inspection same as the inspection performed for the patient of the prediction target from the prediction data DB 36 and generates a learned model using the read out prediction data. The learned model is a model that is used in “machine learning” that is one of fields of artificial intelligence. The learned model includes an algorithm (function) generated from the data stored in the prediction data DB 36 and a parameter tuned in order to increase the prediction accuracy. For example, a certain patient and another patient sometimes have some similarity in a transition pattern of the rate of change of the cell mass before a symptom develops although they are different in both the timing at which the cell mass is measured and the measurement interval. FIG. 9 is a view for explaining about a learned model. However, FIG. 9 does not indicate a learned model itself. For example, as depicted in FIG. 9, the cell mass of the patient A varies within a short period of time, and the cell mass of a patient B varies over a long period of time. However, it is considered that the transition patterns of the rate of change of the cell mass of the patients A and B are similar in their characteristic that the cell amount increases once and then decreases, whereafter it suddenly increases. Although the description here is given of an example that includes two patients, the knowledge that, in the case where a similar characteristic is found among many patients, in the index of the cell mass, the characteristic suggests the possibility of development of tuberculosis is obtained by learning. Accordingly, the prediction unit 28 generates a learned model for predicting a future state of a patient by learning a characteristic common to the rates of change of a specific index from data collected from a plurality of patients. The prediction data read out from the prediction data DB 36 by the prediction unit 28 preferably is successive pieces of data in the prediction data DB 36 from the point of view of the prediction accuracy.

The learned model may be generated at a timing at which a new piece of data is stored into the prediction data DB 36 and stored in advance into a given storage region. In this case, at S52, the prediction unit 28 may read out the learned model corresponding to the patient of the prediction target from the storage region.

Then at S54, the prediction unit 28 acquires an inspection result of the patient of the prediction target from the inspection result table 30. Then, the prediction unit 28 predicts a future state of the patient of the prediction target based on the acquired inspection result and the generated learned model. For example, by applying the rate of change of the cell mass of the patient of the prediction target to the learned model, it may be predicted whether the symptom of tuberculosis develops in the future, in the case where the symptom of tuberculosis develops, when it develops, and so forth.

Then at S56, the prediction unit 28 outputs a result of the prediction. For example, the prediction unit 28 transmits the prediction result to the doctor terminal 70. Consequently, the doctor who uses the doctor terminal 70 may confirm the prediction result of the future state of the patient of the prediction target, and therefore, it is possible for the doctor to send an appropriate advice to the patient or carry out an appropriate treatment.

While the foregoing description is directed to a case in which the prediction unit 28 has the two functions (function for creating a learned model and function for performing prediction using the learned model), the two functions may not necessarily be provided in the prediction unit 28. For example, the functions may be provided in different prediction units (for example, in a first prediction unit and a second prediction unit). The prediction unit 28 may be provided in an apparatus different from the prediction server 12. The two functions the prediction unit 28 has may be provided in different apparatus from each other. In this case, one of the functions the prediction unit 28 has may be provided in the prediction server 12.

As described in detail above, according to the present embodiment, the designation acceptance unit 20 accepts a designation of an inspection item (S12), and the data acquisition unit 22 acquires inspection results and inspection date and time corresponding to the inspection item from the inspection result table 30 (S14). Then, the data exclusion unit 24 refers to the period master 34 and the intervention table 32 to specify an inspection result to be used for generation of prediction data from among the acquired inspection results (S20 to S34), and the prediction data generation unit 26 calculates a rate of change of the inspection result for each patient based on the specified inspection result to generate prediction data (S36). Consequently, in the present embodiment, by taking medical data separate from the inspection result (data of the intervention table 32) into account, it is possible to exclude inspection results that are inappropriate as inspection results to be used for generation of prediction data. Consequently, it is possible to generate prediction data suitable to generate a learned model that is used when a future state of the patient is predicted. Since the prediction data is data indicative of a transition (change) of the rate of change of the inspection result for each patient, a mismatch in inspection interval among different patients may be absorbed. For example, in the medical field, data is acquired after every given interval (first time, one week later, two weeks later, one month later, for example), and even if it is tried to learn a tendency of the inspection results based on the data, if the inspection interval differs among different patients, it is difficult to collect data and to perform learning with a high degree of accuracy. However, if a rate of change of an inspection result is determined as prediction data as in the present embodiment, even if the inspection interval differs, learning with a high degree of accuracy may be performed. In the present embodiment, since a rate of change of an inspection result is used as prediction data, even if some inspection results are excluded based on the period master 34, appropriate prediction data may be generated.

While the present embodiment is directed to a case in which the prediction data generation unit 26 determines a rate of change of an inspection result and uses data indicative of a transition of the rate of change as prediction data, the prediction data is not limited to this. For example, the prediction data generation unit 26 may determine an amount of change of an inspection result and use data indicative of a transition of the amount of change as prediction data. Even with this, the prediction unit 28 may generate a learned model from the transition data of the amount of change and predict a future state of the patient of the prediction target with a high degree of accuracy based on the generated learned model.

In the present embodiment, at S12, the designation acceptance unit 20 accepts a designation of a symptom of a patient, and at S14, the data acquisition unit 22 acquires an inspection result and inspection date and time of a patient, in whom the designated symptom has developed, corresponding to the designated inspection item from the inspection result table 30. Consequently, by generating a learned model based on the inspection result of the patient in whom the symptom has developed, it is possible to predict whether the symptom develops in the patient of the prediction target and, in the case where the symptom develops, when the symptom development time is.

While the embodiment described hereinabove is directed to a case in which a learned model is generated using an inspection result of a patient in whom a designated symptom has developed, generation of a learned model is not limited to this. For example, a learned model may be generated using an inspection result of a patient who indicates an inspection result that reaches a certain numerical value. In this case, it may be predicted at which time the inspection result of the patient of the prediction target is to reach a certain numerical value or the like.

(Modifications)

While the embodiment described above is directed to a case in which, for a period of time defined in the period master 34 after a certain treatment is performed, an inspection result corresponding to the treatment is not used for generation of prediction data, the case in which the inspection result is not used is not limited to this. For example, only in the case where the inspection result corresponding to the treatment indicates a given tendency of the variation, the inspection result corresponding to the treatment may be suppressed from being used for generation of prediction data for a period of time defined in the period master 34.

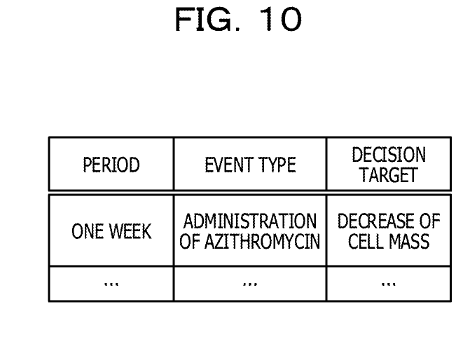

FIG. 10 is a view depicting a modification to the period master. For example, it is assumed that “decrease of cell mass” is stored in the field for “decision target” in the period master as depicted in FIG. 10. In this case, the data exclusion unit 24 suppresses, only in the case where the cell mass decreases after administration of azithromycin, use of inspection results obtained within a period of one week after the administration for generation of prediction data. Accordingly, in the case where the cell mass does not decrease after the administration of azithromycin (in the case where the cell mass does not change or increases), the data exclusion unit 24 uses even an inspection result obtained within the period of one week after the administration for generation of prediction data. This makes it possible to use, even after a treatment, such an inspection result on which an influence of the treatment does not appear for generation of prediction data.

In the present embodiment, information of a patient other than a patient ID, for example, information of the sex, age and so forth of the patient, may be stored in advance into the prediction data DB 36 (FIG. 7). In this case, when the prediction unit 28 generates a learned model, it may use only prediction data of a patient similar to the patient of the prediction target (for example, a patient who is same in sex and similar in age).

While the embodiment described above is directed to a case in which data managed in the electronic medical record server 10 is replicated into the inspection result table 30 and the intervention table 32 of the prediction server 12, the prediction server 12 is not limited to this. For example, the prediction server 12 may directly read out data of inspection results or data relating to treatments, which are managed in the electronic medical record server 10 (stored in the electronic medical record DB), from the electronic medical record DB.

The processing functions described above may be implemented by a computer. In this case, a program that describes the processing substance of the functions the processing apparatus is to have is provided. By executing the program on the computer, the processing functions described above are implemented on the computer. The program that describes the processing substance may be recorded into a computer-readable recording medium (except a carrier wave) in advance.

In the case where the program is to be distributed, it is sold, for example, in the form of a portable recording medium such as a digital versatile disc (DVD) or a compact disc read only memory (CD-ROM) on which the program is recorded. Also it is possible to store the program into a storage apparatus of a server computer in advance such that the program is transferred from the server computer to a different computer through a network.

The computer that executes the program stores, for example, the program recorded on a portable recording medium or the program transferred from the server computer into an own storage apparatus. Then, the computer reads the program from the own storage apparatus and executes processing in accordance with the program. The computer may read out the program directly from the portable recording medium and execute processing in accordance with the program. Also it is possible for the computer to execute, every time a program is transferred from the server computer, a process in accordance with the received program.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A non-transitory computer-readable storage medium storing a program that causes a computer to execute a process, the process comprising:
accepting a designation of an inspection item;
acquiring, from inspection result information in which inspection results and inspection date and time of a patient are stored, inspection results and inspection date and time corresponding to the designated inspection item;
referring to period information that defines a period within which, when a treatment is performed for the patient, an inspection result of an inspection item corresponding to the treatment is not used for generation of prediction data, intervention information in which a processing substance carried out for the patient and treatment date and time are stored, and inspection date and time for each of the acquired inspection results, and specifying inspection results to be used for generation of the prediction data from among the acquired inspection results; and
generating the prediction data based on the specified inspection results.

2. The storage medium according to claim 1, wherein the generating includes
generating at least one of a rate of change of the inspection results and an amount of change of the inspection results for each patient as the prediction data.

3. The storage medium according to claim 1, wherein the specifying includes
specifying, where a treatment is performed for the patient and the inspection results of the inspection item corresponding to the treatment do not indicate a tendency of a change determined in advance, an inspection result within the period that is defined by a definition unit and within which the inspection result is not used for generation of the prediction data as the inspection result to be used for generation of the prediction data.

4. The storage medium according to claim 1, further comprising:
accepting a designation of a symptom of the patient,
wherein the acquiring includes
acquiring an inspection result and inspection date and time, which correspond to the inspection item, of the patient in whom the designated symptom develops, from the inspection result information.

5. The storage medium according to claim 4, wherein the acquiring includes:
specifying identification information for identifying the patient based on information included in the designation of a symptom of the patient, and
acquiring the inspection result and the inspection date and time corresponding to the specified identification information from the inspection result information.

6. The storage medium according to claim 1, wherein the generating includes
calculating at least one of the rate of change and the amount of change in regard to successive inspection results from among the inspection results specified for each patient.

7. The storage medium according to claim 1, further comprising:
generating, based on the prediction data of a plurality of patients including the patient, a model indicative of a characteristic of the prediction data;
predicting a future state of a designated different patient using the model; and
outputting a result of the prediction.

8. The storage medium according to claim 7,
wherein the plurality of patients are patients who undergo an inspection that has been undergone by the different patient, and
the model is a model that relates to at least one of the rate of change and the amount of change in regard to a specific inspection item obtained by a specific inspection.

9. A data generation method executed by a processor of a data generation apparatus, the data generation method comprising:
accepting a designation of an inspection item;
acquiring, from inspection result information in which inspection results and inspection date and time of a patient are stored, inspection results and inspection date and time corresponding to the designated inspection item;
referring to period information that defines a period within which, when a treatment is performed for the patient, an inspection result of an inspection item corresponding to the treatment is not used for generation of prediction data, intervention information in which a processing substance carried out for the patient and treatment date and time are stored, and inspection date and time for each of the acquired inspection results, and specifying inspection results to be used for generation of the prediction data from among the acquired inspection results; and
generating, based on the specified inspection results, at least one of a rate of change of the inspection results and an amount of change of the inspection results for each patient as the prediction data.

10. A data generation apparatus, comprising:
a memory; and
a processor coupled to the memory and configured to:
accept a designation of an inspection item;
acquire, from inspection result information in which inspection results and inspection date and time of a patient are stored, inspection results and inspection date and time corresponding to the designated inspection item;
refer to period information that defines a period within which, when a treatment is performed for the patient, an inspection result of an inspection item corresponding to the treatment is not used for generation of prediction data, intervention information in which a processing substance carried out for the patient and treatment date and time are stored, and inspection date and time for each of the acquired inspection results, and specify inspection results to be used for generation of the prediction data from among the acquired inspection results; and
generate, based on the specified inspection results, at least one of a rate of change of the inspection results and an amount of change of the inspection results for each patient as the prediction data.

11. The data generation apparatus according to claim 10, wherein the processor is configured to
specify, where a treatment is performed for the patient and the inspection results of the inspection item corresponding to the treatment do not indicate a tendency of a change determined in advance, an inspection result within the period that is defined by a definition unit and within which the inspection result is not used for generation of the prediction data as the inspection result to be used for generation of the prediction data.

12. The data generation apparatus according to claim 10, wherein the processor is configured to:
accept a designation of a symptom of the patient, and
acquire an inspection result and inspection date and time, which correspond to the inspection item, of the patient in whom the designated symptom develops, from the inspection result information.

13. The data generation apparatus according to claim 12, wherein the processor is configured to:
specify identification information for identifying the patient based on information included in the designation of a symptom of the patient, and
acquire the inspection result and the inspection date and time corresponding to the specified identification information from the inspection result information.

14. The data generation apparatus according to claim 10, wherein the processor is configured to
calculate at least one of the rate of change and the amount of change in regard to successive inspection results from among the inspection results specified for each patient.

15. The data generation apparatus according to claim 10, wherein the processor is configured to:
generate, based on the prediction data of a plurality of patients including the patient, a model indicative of a characteristic of the prediction data;
predict a future state of a designated different patient using the model; and
output a result of the prediction.

16. The data generation apparatus according to claim 15,
wherein the plurality of patients are patients who undergo an inspection that has been undergone by the different patient, and
the model is a model that relates to at least one of the rate of change and the amount of change in regard to a specific inspection item obtained by a specific inspection.
